(19) United States
(12) Patent Application Publication
(10) Pub. No.: US 2008/0056691 A1
(43) Pub. Date: Mar. 6, 2008

(54) Vapor dispenser with indicator

(76) Inventors: James P. Wingo, Peachtree City, GA (US); Edward M. Kaucic, Newnan, GA (US); Ike Haldopoulos, Fairburn, GA (US); Mark Albert Peterson, Vidalia, GA (US); Giorgos Hatzilias, Buford, GA (US); John Taylor JR., Dacula, GA (US)

Correspondence Address: KILPATRICK STOCKTON LLP, 1001 WEST FOURTH STREET, WINSTON-SALEM, NC, 27101, US

(21) Appl. No.: 11/805,204

(22) Filed: May 21, 2007

Related U.S. Application Data
(60) Provisional application No. 60/801,997, filed on May 19, 2006.
(60) Provisional application No. 60/853,242, filed on Oct. 20, 2006.

(51) Int. Cl. A61L 9/04; A61L 9/02; G08B 21/00; H05B 3/06

(52) U.S. Cl. 392/395; 222/23; 222/39; 239/35; 29/428; 340/618

(57) ABSTRACT

Various embodiments of devices and methods for indicating the level or amount of a vaporizable material in a dispenser, such as an air freshener device or insect control system, are disclosed. An embodiment of a dispenser includes a housing with a reservoir and wick that may be removably coupled to the housing. A circuit within the dispenser measures a property in the wick, such as an electrical property, or a level of vaporizable material in the reservoir and triggers an indicator that provides a visual or audible signal to the user advising that the reservoir is low or empty.

### PRIOR RELATED APPLICATION DATA

This application hereby claims priority to U.S. Provisional Patent Application Ser. No. 60/801,997, filed May 19, 2006 and U.S. Provisional Patent Application Ser. No. 60/853,242, filed Oct. 20, 2006.

### FIELD OF THE INVENTION

The present invention relates to dispensers of vaporizable materials and, in particular, to a fluid level indicator for a dispenser.

### BACKGROUND OF THE INVENTION

Many people place air fresheners in a room to cover up odors in the room or to add a fragrant scent to the air. The need for effectively combating malodors in homes and enclosed public buildings, by odor masking or destruction, is well established, as is the dispensing of insect control materials for killing or deterring insects. Various kinds of vapor-dispensing devices have been employed for these purposes. In particular, wicking devices are well known for dispensing volatile liquids into the atmosphere, such as a fragrance, deodorant, disinfectant, insect repellant, or insecticide active agent. A typical wicking device utilizes a combination of a wick and an emanating region to dispense a volatile liquid from a liquid reservoir. Additionally, wicking devices in which the wicking action is promoted by a heat source are also known.

Many air fresheners are commercially available. Air fresheners that utilize wicking action and are plug-in and/or battery-powered diffusers are particularly popular with consumers. Plug-in diffusers are well known in the art. In these devices, a resistance heater is disposed in a housing, out of which electrical prongs extend directly. When the prongs are plugged into a wall socket, the resistance heater generates heat. A substance, such as a fragrance or an insect repellant, to be emitted into the air is maintained, typically in liquid form, in close proximity to the heater. As the heater heats the substance, controlled amounts are vaporized and emitted into the surrounding atmosphere. These devices are particularly well suited for domestic use, especially in rooms such as kitchens and bathrooms, because they provide a continuous, controlled flow of a desired substance into the air. Battery-powered diffusers are also well known and function in a substantially similar manner, except that the unit is powered by a consumer-grade battery cell rather than electricity from a wall outlet and thus prongs for plugging into the wall and AC-DC power conversion circuitry may not be necessary in battery-powered units.

Once someone initially places the air freshener in a room that person typically forgets about the air freshener. Thus, after extended use, air fresheners often go empty for some time without being noticed. This may be attributed, in part, to the subtly of the gradual decline in scent as well as a person's adaptation to the scent. In other words, people are unable to detect when the air freshener is empty based on lack of perception the scent alone, and they need some other sensory clue indicating that it is time for a new air freshener or to replace a replaceable liquid reservoir of the air freshener.

Many commercial air fresheners include features beyond simply providing a fresh scent. For example, combination air freshener/night lights, fragrance boost buttons that engage a small fan for a few seconds, and dual fragrance emitters that alternate fragrances are all commercially available. However, none of these or other commercially available devices provides an indication to the user when the liquid level in the device is low or empty and needs to be refilled or replaced. Moreover, circuits and sensing mechanisms in such air fresheners are relatively complex and utilize numerous components.

In view of the foregoing, a need exists for devices comprising indicators operable to provide users with a signal advising that levels or amounts of a vaporizable material in the device are low or depleted. Additionally, there is a need for devices that provide more simple, efficient sensing circuitry for use in plug-in and battery-powered air fresheners.

### SUMMARY

The present invention provides devices and methods for indicating the level or amount of a vaporizable material in a dispenser. Devices and methods of the present invention can alert a user that the level or amount of vaporizable material in the device is low or depleted. In some embodiments, a vaporizable material is a liquid. In other embodiments, a vaporizable material is a gel, paste, or a solid such as, but not limited to, a wax. Vaporizable materials, in some embodiments of the present invention, comprise fragrances. In another embodiment, vaporizable materials comprise deodorants, disinfectants, insect repellants, or insecticide active agents.

In one embodiment, the present invention provides a dispenser of vaporizable material, the dispenser comprising a housing; a reservoir coupled to the housing, the reservoir containing a vaporizable material and a wick at least partially disposed in the reservoir; a circuit configured to measure a property in the wick or measure the vaporizable material in the reservoir; and an indicator triggered by the circuit if a measurement is above, below, or equal to a predetermined threshold. Indicators, in some embodiments of the present invention, can provide a visual or audible signal indicating that the level or amount of vaporizable material in the dispenser is low. Moreover, the property measured in the wick may be, for example, conductivity, capacitance, dielectric change, inductance, temperature, or any other suitable property that can vary based on the amount of vaporizable material in the wick. In one embodiment, for example, the measurable property can vary as a function of the wetness of the wick.

In some embodiments, the indicator provides an active signal that alerts the user when the vaporizable material in the dispenser is empty or needs to be replaced. By providing an active signal to the user, the dispenser provides valuable information to the consumer in an effective manner that is easy for the consumer to understand and makes it simple for the consumer to know when the dispenser needs to be changed or refilled. Certain embodiments of this invention sense the presence or absence of a vaporizable material within a reservoir or wick in various manners in order to trigger an indicator, such as an LED light. Some examples include sensing a change in electric current, voltage, or other property across a wet wick versus a dry or almost dry wick; using an electronic eye to detect a difference in or lack of light when a vaporizable material, such as a liquid, is present; using the presence or absence of a light reflection on the surface of a liquid; causing a change in signal reflected when a change in wetness in an RFID (radio frequency identification) tag is detected, and several others further described below.

In another aspect, the present invention provides methods of making a dispenser. A method of making a dispenser, in one embodiment, comprises providing a housing; coupling a reservoir to the housing, the reservoir containing a vaporizable material and a wick at least partially disposed in the reservoir; providing a circuit configured to measure a property in the wick or measure the vaporizable material in the reservoir; and coupling the circuit to an indicator operable to provide a signal if a measurement is above, below, or equal to a predetermined threshold.

In a further aspect, the present invention provides methods of indicating the level of a vaporizable material in a dispenser comprising providing a circuit; obtaining a measurement of a property in a wick of the dispenser or a measurement of the amount of vaporizable material in a reservoir of the dispenser with the circuit; coupling the circuit to an indicator; and providing a signal with the indicator based upon the value of the measurement. In some embodiments, the value of the measurement obtained by the circuit is greater than a predetermined threshold value. In other embodiments, the value of the measurement is less than a predetermined threshold value. In a further embodiment, the value of the measurement obtained by the circuit is equal to a predetermined threshold value. In one embodiment, the signal is an audible and/or visual signal indicating that the level or amount of vaporizable material in the dispenser is low or depleted.

These and other embodiments are described in greater detail in the following detailed description of the disclosed embodiments and claims.

### BRIEF DESCRIPTION OF THE FIGURES

FIG. 1 is a perspective view of one embodiment of a dispenser according to the present invention.

FIG. 2 is a perspective view of the dispenser of FIG. 1 with the reservoir removed from the housing.

FIG. 3 is an exploded view of the dispenser of FIG. 1.

FIG. 4 is a perspective view of the sensing unit shown in FIG. 3.

FIG. 5 is a perspective view of another embodiment of a dispenser according to the present invention.

FIG. 6 is an exploded view of the dispenser of FIG. 5.

FIG. 7 is a partial view of an electronic eye emitter and receiver of the dispenser of FIG. 5.

FIG. 8 is a perspective view of another embodiment of a dispenser of this invention.

FIG. 9 is an exploded view of the dispenser of FIG. 8.

FIG. 10 is an exemplary embodiment of an AC-DC power converter circuit useful in certain embodiments of this invention.

FIG. 11 is an exemplary embodiment of a multi-transistor, inverter circuit useful in certain embodiments of this invention.

FIG. 12 is an exemplary embodiment of a circuit useful in certain embodiments of this invention.

FIG. 13 is an exemplary embodiment of a circuit useful with certain embodiments of dispensers according to the present invention that use both AC mains power and battery power.

### DETAILED DESCRIPTION

The present invention provides devices and methods for indicating the level or amount of a vaporizable material in a dispenser. Devices and methods of the present invention can alert a user that the level or amount of vaporizable material in the device is low or depleted.

In one embodiment, the present invention provides a dispenser of vaporizable material, the dispenser comprising a housing; a reservoir coupled to the housing, the reservoir containing a vaporizable material and a wick at least partially disposed in the reservoir; a circuit configured to measure a property in the wick or measure the vaporizable material in the reservoir; and an indicator triggered by the circuit if a measurement is above, below, or equal to a predetermined threshold.

I. Dispenser

A dispenser, in some embodiments, may be a plug-in, battery-powered, or combination plug-in/battery-powered air freshener or room deodorizer and the vaporizable material level indicator provides a signal when a replaceable reservoir of vaporizable material is low or empty and needs to be replaced. In one embodiment, there is an electrical plug with contact blades that extend from the housing and are configured to plug into any 110 or 240 volt electrical outlet. In an alternative embodiment, a plug may be configured for plugging into an automobile or other device with a 12 V power supply.

One embodiment of a circuit within a housing of a dispenser for use in a standard electrical outlet includes an AC-DC power converter, as well as circuitry to measure a property in the wick or to measure a level of a vaporizable material, such as a liquid, in the reservoir. Certain embodiments that use consumer-grade battery cells, instead of power from an electrical outlet, will not include AC-DC power converter circuitry. In one embodiment, a plug-in air freshener for 110-240 V outlets has its heating element or fan powered by AC mains power, and also includes an independent battery-powered circuit for the dispenser's sensing unit that measures electrical properties in or across the wick and controls an indicator that signals when the dispenser is low or empty.

In another embodiment, the dispenser is entirely battery-powered. In certain embodiments, a circuit within the housing triggers an indicator, for example, when the level or amount of vaporizable material is above or below a predetermined threshold, for example, when there is no longer any vaporizable material remaining in the reservoir or when there is no longer any current conducting through the wick (indicating a dry wick).

II. Indicators

Indicators of a dispenser, in some embodiments of the present invention, can provide a visual or audible signal indicating that the level or amount of vaporizable material in the dispenser is low. In other embodiments, indicators can provide a visual or audible signal indicating that the level of vaporizable material in the dispenser is at a sufficient or full level.

In some embodiments, an indicator provides an active signal that alerts the user when the vaporizable material in the dispenser is empty or needs to be replaced. By providing an active signal to the user, the dispenser provides valuable information to the consumer in an effective manner that is easy for the consumer to understand and makes it simple for the consumer to know when the dispenser needs to be changed or refilled. Certain embodiments of this invention sense the presence or absence of a vaporizable material within a reservoir or wick in various manners in order to trigger an indicator, such as an LED light. Some examples include sensing a change in electric current, voltage, or other property across a wet wick versus a dry or almost dry wick; using an electronic eye to detect a difference in or lack of light when a vaporizable material, such as a liquid, is present; using the presence or absence of a light reflection on the surface of the liquid; causing a change in signal reflected when a change in wetness in an RFID tag is detected, and several others further described below.

In certain embodiments, the indicator may be any visual or audible signal, such as a light on, light off, light blinking or flashing, light changing color, one-time sound, repeating sound, etc. An RFID tag could also be included to provide remote communication of the indicated status of the level of vaporizable material, as further described below. As provided herein, the wick may be synthetic fiber, porous plastic, or cellulosic material. A dispenser may also include a fan or heating element, and the reservoir and wick may be removable from the housing of the dispenser. In an embodiment with an LED indicator, the LED indicator could also be used as a nightlight. Such a device may also have a built-in photosensor that can automatically turn the indicator on or off based on the ambient light intensity when the fragrant level is a certain threshold or does not need to be refilled or changed. Once the level or amount of vaporizable material is below a threshold or needs to be refilled or changed, a circuit prevents the photosensor from automatically turning on or off the indicator, and the indicator remains on regardless of the ambient light intensity from the surrounding environment.

III. Vaporizable Material

In some embodiments, a vaporizable material is a liquid. In other embodiments, a vaporizable material is a gel, paste, or a solid such as, but not limited to, a wax. Vaporizable materials, in some embodiments of the present invention, comprise fragrances. In another embodiment, vaporizable materials comprise deodorants, disinfectants, insect repellants, or insecticide active agents.

In some embodiments wherein a vaporizable material is a gel, the gel can be constructed by mixing a fragrance, deodorant, disinfectant, insect repellant, and/or insecticide agent with an aqueous based solution and a gel forming agent, such as carrageenan and/or carboxymethylcellulose (CMC). In another embodiment, a fragrance, deodorant, disinfectant, and/or insecticide is mixed with an alcohol based solution and a gel forming agent in the production of a vaporizable gel material.

Additionally, in some embodiments wherein a vaporizable material is a solid, the solid can be constructed by mixing a fragrance, deodorant, disinfectant, insect repellant, and/or insecticide with a liquid wax and subsequently cooling the mixture to solid form. In one embodiment, the mixture is sprayed prior to cooling to form a powder. Waxes suitable for use in solid vaporizable materials can comprise a natural wax, such as hydroxystearate wax, or a petroleum based wax, such as a paraffin. In some embodiments, polyethylene oxide (PEO) is used as a substrate for a fragrance, deodorant, disinfectant, insect repellants and/or insecticide.

Vaporizable fragrances, disinfectants, deodorants, insect repellants, and insecticides are well known to one of skill in the art and are available from a variety of commercial sources. Common fragrances comprise citrus oils, fruity floral oils, herbal floral oils, lemon oils, orange oils, or combinations thereof. Disinfectants, in some embodiments, comprise denatonium benzoate, hinokitiol, benzthiazolyl-2-thioalkanoic nitrites, alkyl dimethylbenzyl ammonium chlorides, or trichlosan. Insect repellants, in some embodiments, comprise N,N-diethyl-meta-toluamide, citronella oils, or camphor. Additionally, insecticides, in some embodiments, comprise imiprotrin, cypermethrin, bifentrint, or pyrethrins.

Vaporizable materials, in some embodiments, are disposed in a reservoir of the dispenser. In one embodiment, a vaporizable material comprises a liquid. As described herein, a liquid vaporizable material can be transported from the reservoir through the wick to a heating element for subsequent vaporization or evaporation. In other embodiments, a vaporizable material is disposed on a surface of the wick or otherwise impregnated into the wick. In such embodiments, the wick serves as the reservoir for the vaporizable material. In one embodiment, for example, a wick is impregnated and/or coated with a solid vaporizable material, such as a wax. In another embodiment, a wick is impregnated and/or coated with a vaporizable material comprising a gel or paste. In some embodiments wherein the wick is impregnated and/or coated with a solid, gel, or paste vaporizable material, the wick serves as a reservoir for the solid, gel, or paste vaporizable material.

IV. Wicks

Wicks, in some embodiments of the present invention, comprise porous plastics including, but not limited to, sintered porous plastics. Porous plastics suitable for use as wicks, according to embodiments of the present invention, comprise thermoplastics, thermosets, elastomers, or combinations thereof.

In another embodiment, a wick comprises a fibrous material. Fibrous materials, according to some embodiments, comprise monocomponent fibers, bicomponent fibers, or combinations thereof. Monocomponent fibers suitable for use in embodiments of the present invention, in some embodiments, comprise polyethylene, polypropylene, polystyrene, nylon-6, nylon-6,6, nylon 12, copolyamides, polyethylene terephthalate (PET), polybutylene terephthalate (TBP), co-PET, or combinations thereof.

Bicomponent fibers suitable for use in wicks, according to some embodiments of the present invention, comprise polypropylene/polyethylene terephthalate (PET); polyethylene/PET; polypropylene/Nylon-6; Nylon-6/PET; copolyester/PET; copolyester/Nylon-6; copolyester/Nylon-6,6; poly-4-methyl-1-pentene/PET; poly-4-methyl-1-pentene/Nylon-6; poly-4-methyl-1-pentene/Nylon-6,6; PET/polyethylene naphthalate (PEN); Nylon-6,6/poly-1,4-cyclohexanedimethyl (PCT); polypropylene/polybutylene terephthalate (PBT); Nylon-6/co-polyamide; polylactic acid/polystyrene; polyurethane/acetal; and soluble copolyester/polyethylene. Biocomponent fibers, in some embodiments, comprise those disclosed in U.S. Pat. Nos. 4,795,668; 4,830,094; 5,284,704; 5,509,430; 5,607,766; 5,620,641; 5,633,032; and 5,948,529.

Bicomponent fibers, according to some embodiments of the present invention, have a core/sheath or side by side cross-sectional structure. In other embodiments, bicomponent fibers have an islands-in-the-sea, matrix fibril, citrus fibril, or segmented pie cross-sectional structure. Bicomponent fibers comprising core/sheath cross-sectional structure and suitable for use in embodiments of the present invention are provided in Table I.

TABLE I
Bicomponent Fibers

| Sheath | Core |
|---|---|
| polyethylene (PE) | polypropylene (PP) |
| ethylene-vinyl | polypropylene (PP) |
| acetate copolymer (EVA) | |
| polyethylene (PE) | polyethylene terephthalate (PET) |
| polyethylene (PE) | polybutylene terephthalate (PBT) |
| Polypropylene (PP) | polyethylene terephthalate (PET) |
| Polypropylene (PP) | polybutylene terephthalate (PBT) |
| polyethylene (PE) | Nylon-6 |
| polyethylene (PE) | Nylon-6,6 |
| polypropylene (PP) | Nylon-6 |
| polypropylene (PP) | Nylon-6,6 |
| Nylon-6 | Nylon-6,6 |
| Nylon-12 | Nylon-6 |
| copolyester (CoPET) | polyethylene terephthalate (PET) |
| copolyester (CoPET) | Nylon-6 |
| copolyester (CoPET) | Nylon-6,6 |
| glycol-modified PET | polyethylene terephthalate (PET) |
| (PETG) | |
| polypropylene (PP) | poly-1,4-cyclohexanedimethyl (PCT) |
| polyethylene | poly-1,4-cyclohexanedimethyl (PCT) |
| terephthalate (PET) | |
| polyethylene | polyethylene naphthalate (PEN) |
| terephthalate (PET) | |
| Nylon-6,6 | poly-1,4-cyclohexanedimethyl (PCT) |
| polylactic acid (PLA) | polystyrene (PS) |
| polyurethane (PU) | acetal |

In some embodiments, fibers comprise continuous fibers. In other embodiments, fibers comprise staple fibers. In one embodiment, for example, a fiber of a fibrous material comprises a staple bicomponent fiber. Staple fibers, according to some embodiments, have any desired length. In some embodiments, fibrous materials are woven or non-woven. In one embodiment, a fibrous material is sintered.

In one embodiment, a wick has an average pore size ranging from about 5 μm to about 500 μm or from about 10 μm to about 400 μm. In another embodiment, a wick comprising a sintered porous plastic has an average pore size ranging from about 50 μm to about 300 μm, from about 100 μm to about 250 μm, or from about 150 μm to about 200 μm. Additionally, a wick, in some embodiments, has a porosity of at least about 30%. In another embodiment, a wick has a porosity ranging from about 30% to about 90%, from about 40% to about 80%, or from about 50% to about 70%. In a further embodiment, a wick has a porosity greater than 90%.

Wicks, according to embodiments of the present invention, can have any desired shape including, but not limited to, cylindrical, conical, triangular, square, tubular, rectangular, polygonal, or star shaped.

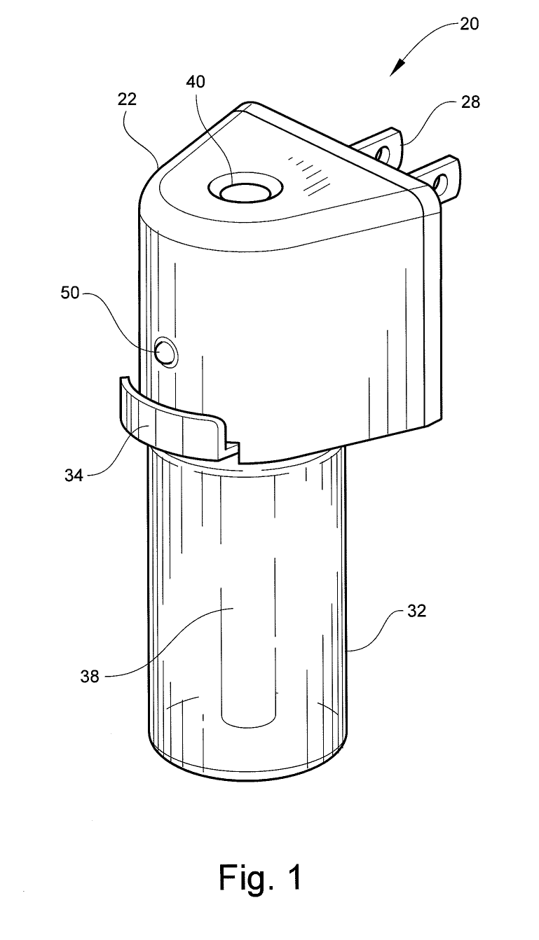

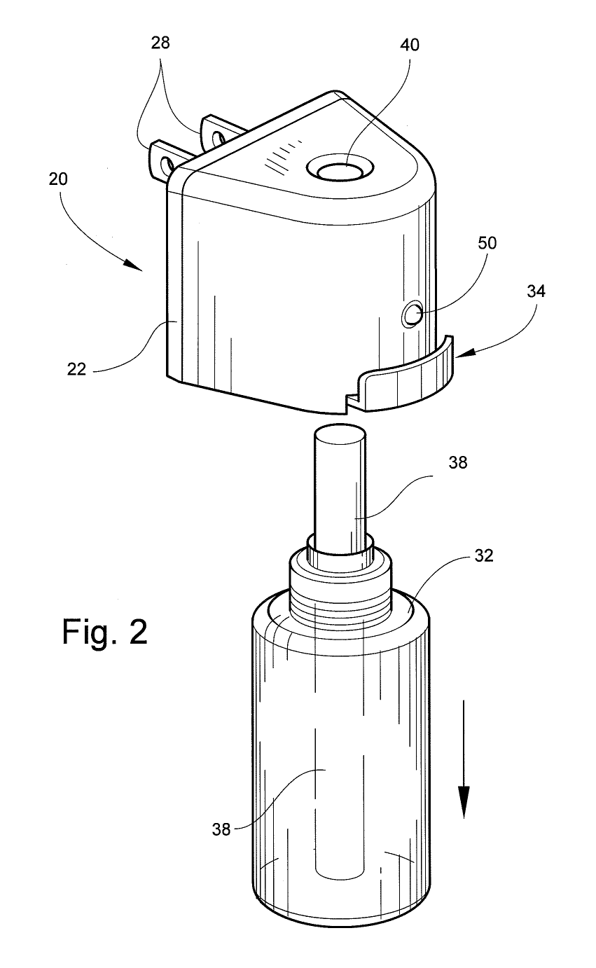

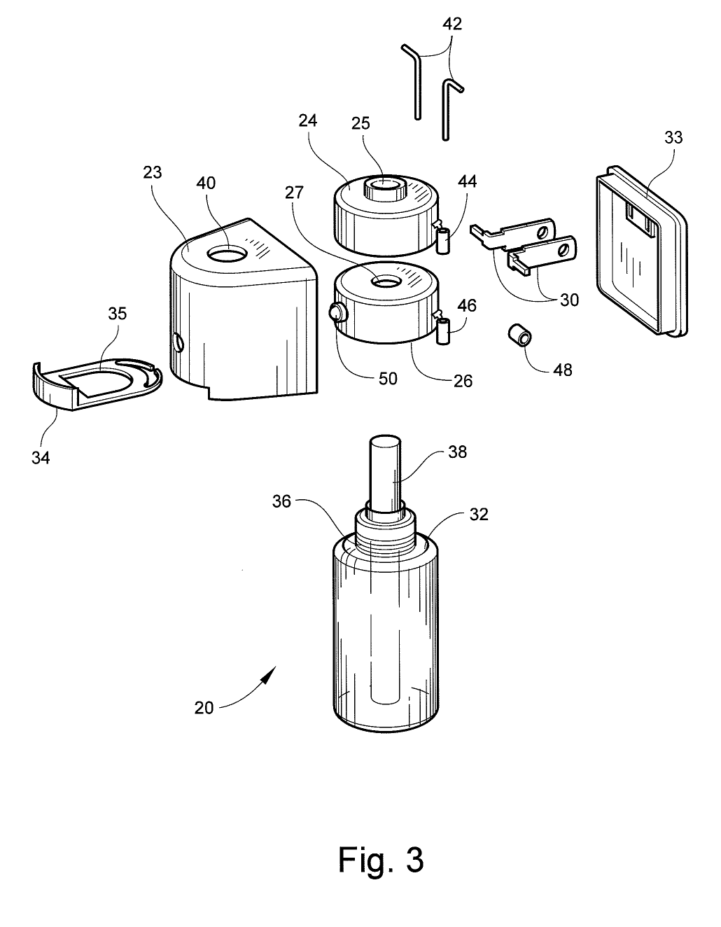

Referring now to the figures, an embodiment of a dispenser 20 is shown in FIGS. 1-4. Dispenser 20 includes a housing 22 with a heating element 24 and a sensing unit 26 positioned therein. Housing 22 includes a central body 23 and a back wall 33, as shown in FIG. 3. Dispenser 20 includes an electrical plug 28 with two contact blades 30 for plugging dispenser 20 into an electrical outlet. A reservoir, jar, or container 32 with liquid therein is coupled to housing 22. In some embodiments, reservoir 32 is releasably coupled to housing 22. A push button/latch 34 is provided in housing 22 for releasably engaging reservoir 32. As shown in FIGS. 2 and 3, reservoir 32 includes a groove 36 that engages an aperture 35 in latch 34. Reservoir 32 also includes a wick 38 that is partially within reservoir 32 and extends partially out of reservoir 32.

Within housing 22, heating element 24 and sensing unit 26 are in a stacked configuration and reservoir 32 attaches to housing 22 such that wick 38 extends through an aperture 27 in sensing unit 26 and with the top end of wick 38 within an aperture 25 in heating element 24. A vaporizable material, such as a liquid, from reservoir 32 flows up into the wick 38 via capillary action. The portion of wick 38 in heating element 24 experiences elevated temperatures, causing the liquid to evaporate and flow out the top of the dispenser 20 through an opening 40 and into the surrounding environment. Heating element 24 and sensing unit 26 are held in place within housing 22 and electrically connected to contact blades 30 using pins 42 and sleeves 44, 46, and 48, which are shown in FIG. 3, as understood by those skilled in the art.

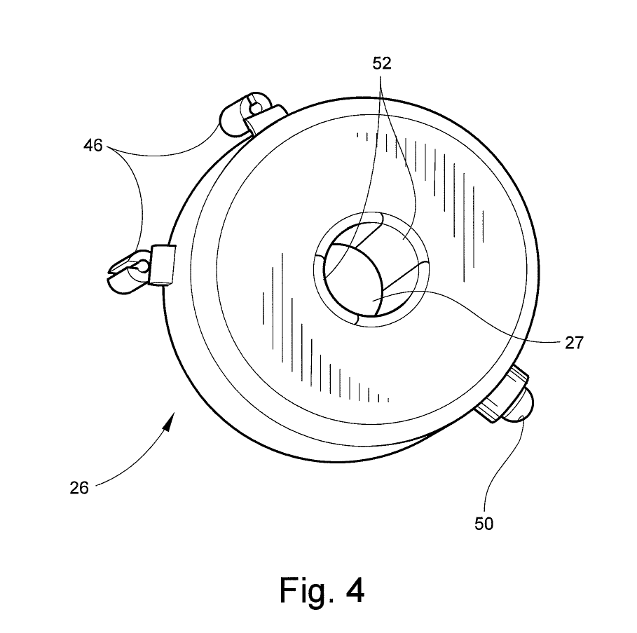

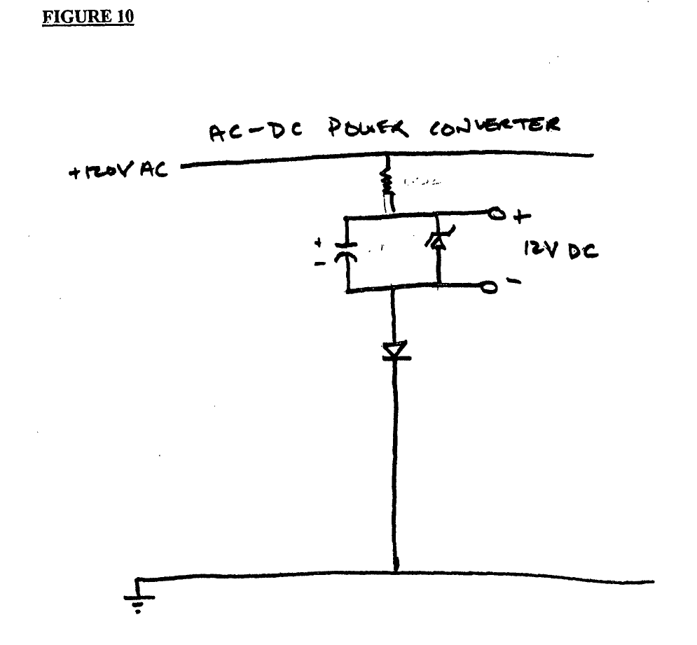

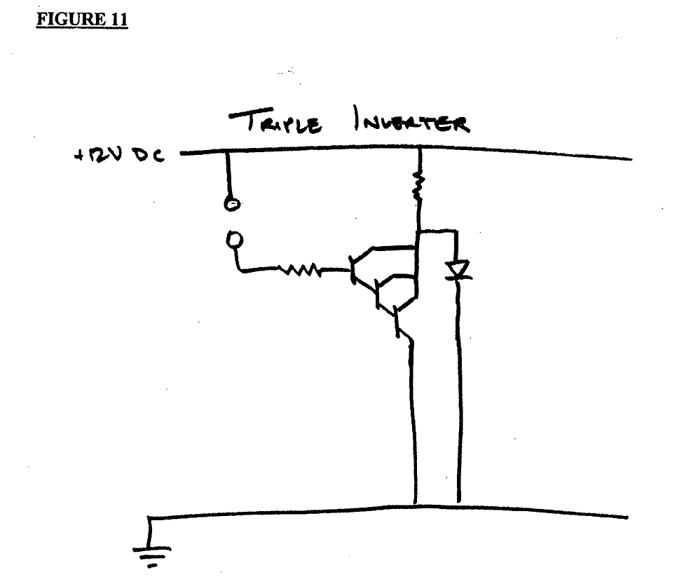

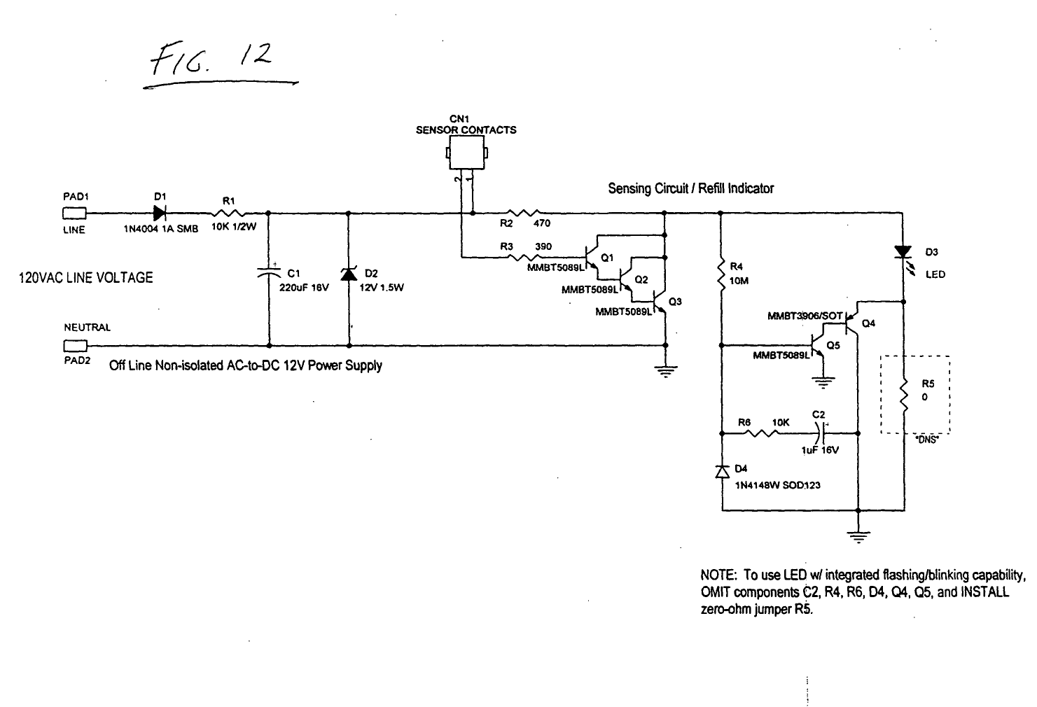

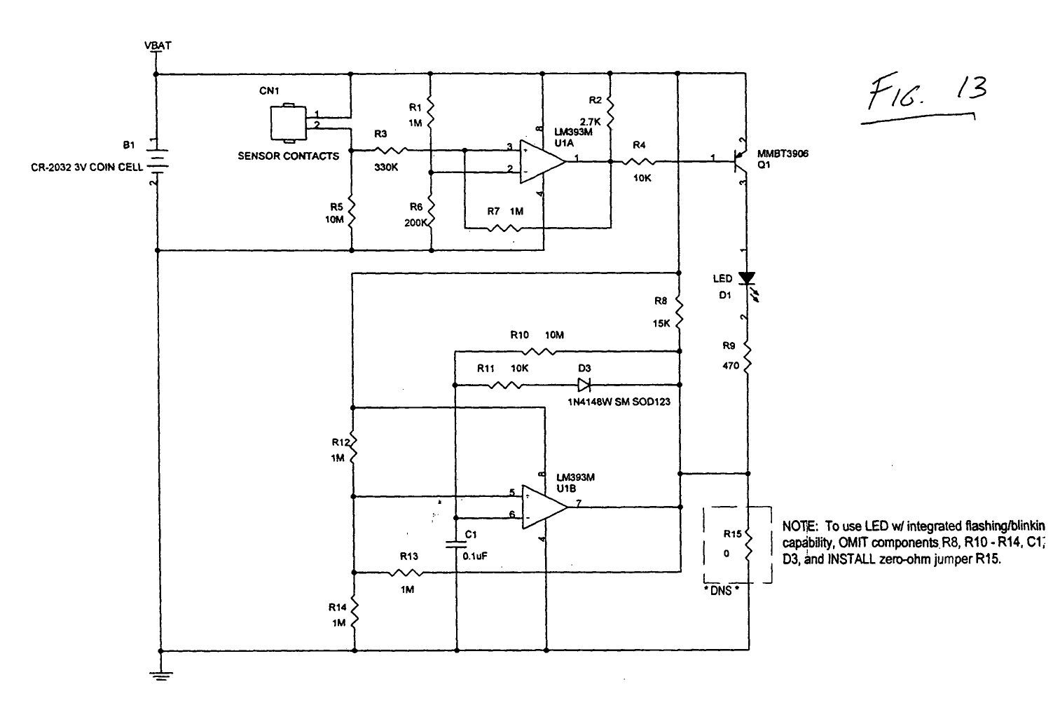

Sensing unit 26, shown in isolation in FIG. 4, includes a light emitting diode or LED 50 and metal contacts 52. Contacts 52 contact the portion of wick 38 that extends through aperture 27. Contacts 52 are connected to circuitry (not shown in FIGS. 1-4) in sensing unit 26 that senses whether wick 38 is wet or less wet based on a property, such as conductivity or voltage, capacitance, inductance, dielectric change, or temperature change. In an exemplary embodiment, the circuitry includes an inverter circuit as shown in FIG. 11 and further described below. In an alternative embodiment, the circuitry includes that shown in FIG. 12, which is described further below. In certain embodiments, the circuitry senses a current through the wick. Some portion of housing 22 also preferably includes an AC-DC power converter circuit, as shown in FIG. 10 and further described below, for providing steady DC power to dispenser 20 and sensing unit 26. In an alternative embodiment, the housing 22 includes a consumer-grade battery circuit, such as that shown in FIG. 13, for providing power to sensing unit 26 and an indicator such as LED 50. Other embodiments may be entirely battery-powered.

When wick 38 begins running dry of vaporizable material or sensing unit 26 senses that the measured property is above or below a predetermined threshold (reflecting that the level or amount of vaporizable material in the reservoir is low or empty), LED 50 is activated to alert the user that a new reservoir of liquid is needed. LED 50 may go from off to on, on to off, off to flashing, on to flashing, change from one color to another, or otherwise provide an indication to the user. To replace reservoir 32, button 34 is depressed, releasing reservoir 32 so that a reservoir with more liquid can be coupled to housing 22.

It should be understood that a predetermined threshold may be arbitrary and may have numerous empirical or other values. For example, a predetermined threshold may simply be set at zero, such that when no current passes from one of contacts 52 to the other of contacts 52 because there is no longer any vaporizable material in wick 38, sensing unit 26 triggers LED 50 or another indicator. The threshold may be set at other than zero to indicate a low, but not empty, liquid level so that the user can be notified to replace the reservoir before the reservoir is entirely empty. Additionally, the threshold may be set, in part, based on the characteristics and tolerances of the circuit components used in sensing unit 26 and/or other components used any similar sensing mechanism described herein. It should be understood that sensing unit 26 and other active sensors described herein merely need to have some mechanism whereby a switch is triggered to activate an indicator based on some property measured or determined by such sensors.

Turning now to FIGS. 10 and 11, exemplary circuit components for use in dispenser 20 are further described. For the embodiment shown in FIGS. 1-4 to work most efficiently and effectively, a steady DC power supply is preferable. For dispensers that are to be used as air fresheners, AC line power may be retrieved from a standard household electrical outlet and converted to DC power using an AC-DC power converter circuit in the housing of the dispenser. This may be preferable because of a lack of space in the dispenser and the inadequacy of some batteries. Although a typical transformer well known to those skilled in the art could be used, such a transformer may not be sufficiently steady, is more costly, and too large to fit within the housing of a typical commercially available air freshener. In some alternative embodiments, however, DC power is provided to the sensing unit and the indicator (but not any heating element or fan present in the dispenser) by a consumer-grade battery cell and a battery circuit, such as that shown in FIG. 13. In such embodiments, AC-DC power conversion is unnecessary. It should be well understood that this invention is not limited to plug-in devices, or devices of a particularly small size, and that a dispenser that is partially or fully powered by a battery is within the scope of this invention.

For use in certain embodiments that are plug-in dispensers, an embodiment of a suitable AC-DC power converter is shown in FIG. 10. This particular embodiment converts 110-120V AC line power to 12V DC power. Typically, an AC-DC power converter may contain resistors, capacitors, rectifying diodes, and/or zener diodes. The embodiment shown in FIG. 10 converter includes a resistor in series with a rectifying diode and a parallel system of a capacitor and a zener diode. In one embodiment, the resistor may be wire wound with a resistance of 6000 ohms, the rectifying diode may be a 1N4007 rectifying diode, the capacitor may be a 22 μF, 50V capacitor, and the zener diode a 12V diode. The output, which is 12V DC, is half-wave rectified, but the capacitor stores energy to the point where the voltage is well stabilized for its intended use. The resistor gives off large amounts of heat to be used to evaporate the liquid more effectively when the AC-DC power converter circuit is used within a heating element, such as heating element 24. Many commercially available air fresheners convert AC line power to DC power, but this embodiment is particularly advantageous because it does so utilizing a simple circuit with few components.

In one embodiment, a circuit in sensing unit 26 is used to determine when vaporizable material is present in the reservoir by passing a current through the wick. Scented oils typically used in air fresheners have very few electrolytes, which may be dissociated into free ions when dissolved in order to provide an electrically conductive medium. Accordingly, a very large current or a very sensitive circuit is required when passing a current through the wick to determine when liquid is present. Because use of a large current would cause unnecessary safety concerns for consumer products such as air fresheners, it is preferable to use a circuit highly sensitive to current change.

One embodiment of a suitable circuit for use in sensing unit 26 is shown in FIG. 11. In some embodiments, light emitting diodes and transistors are used because they are small and relatively inexpensive, with the transistors functioning as switches and amplifiers as will be well understood by those skilled in the art. As shown in FIG. 11, three transistors are used in combination. Although a single transistor may produce a circuit appropriate for passing current through a wick comprising a vaporizable material while two transistors (known as a Darlington transistor or Darlington pair) produce a usable touch switch, the configuration shown in FIG. 11 using three transistors is preferable. The third transistor is coupled to the second transistor by coupling the gate of the third transistor to the emitter of the second transistor (in other words, the same way the first and second transistors of a Darlington pair are coupled to one another). In an alternative embodiment using battery power to power the sensing unit and indicator, shown in FIG. 13, an analog comparator integrated circuit is used to “compare” the voltage at the non-inverting input resulting from current passing through a liquid-wet wick to the reference voltage set by the resistor network connected to the inverting input. The embodiment shown in FIG. 13 is described in further detail below.

The use of three transistors provides a switch with the desired sensitivity for detecting current change across a wick comprising vaporizable material. For example, a single transistor is not sufficiently sensitive to detect the absence/presence of scented oils used in air fresheners. By adding two more transistors in the manner shown, a very small difference in conductivity can be detected by leveraging that small change to flip progressively “bigger” switches, as is well understood by those skilled in the art. In one embodiment, a preferred transistor is a 2N2222 small signal transistor. The circuit shown in FIG. 11 also acts as an inverter so that LED 50 goes on when the wick is dry indicating that the reservoir is empty, rather than going off when the reservoir is empty. In an alternative embodiment, the use of analog comparator circuit powered by battery, such as in the embodiment shown in FIG. 13, provides the desired sensitivity for detecting a change in current across a liquid-wet wick. The circuit shown in FIG. 13 activates LED 50 when the wick is dry (or almost dry) indicating that the reservoir is empty. FIG. 13 is described in further detail below.

Another exemplary embodiment of a suitable circuit for use in sensing unit 26 in a plug-in dispenser is shown in FIG. 12. The embodiment shown in FIG. 12 incorporates a simple non-isolated, AC-to-DC circuit that converts the 120V AC input voltage to a regulated +12V DC supply that powers the sensing/indicator circuitry. The rectifier diode D1 conducts on the positive half of the incoming sinusoidal voltage signal, thereby charging up filter capacitor C1. During the negative half of the waveform, D1 does not conduct and the voltage at capacitor C1 begins to discharge. Zener diode D2 serves as a cost-effective voltage regulator, limiting the DC voltage to 12 volts. The three transistors Q1-Q3 have high-gain characteristics and are configured to sense the extremely low current flowing through the contacts at CN1. As long as sufficient current flows through this sensing circuitry, Q1-Q3 remain in the “on” state, effectively grounding the anode terminal of the indicator LED. Once the current flow drops sufficiently, transistors Q1-Q3 turn off, thereby removing the ground condition from the LED anode terminal. This results in a positive voltage being applied to the anode of the LED, thus enabling the refill indicator. The circuitry comprised of components C2, R4, R6, D4, Q4, and Q5 serves to flash a standard LED intermittently once per second. By adjusting the values of these components, other flash frequencies are possible. To use an LED with an integrated flash or blink capability, this circuitry may be eliminated and replaced by zero-ohm jumper R5.

As noted above, certain embodiments of dispensers may be battery-powered, plug-in, or a combination of plug-in and battery powered. Dispensers with some battery power may be advantageous for several reasons, including that battery-powered devices do not require the same rigorous approval from various safety regulatory agencies (UL, CSA, etc.) as dispensers that are powered entirely by AC mains power. An exemplary embodiment of a circuit suitable in a sensing unit of a dispenser that is partially battery-powered is shown in FIG. 13. In this embodiment, the battery is used to power the sensing unit and indicator, but not a heating element or fan within the dispenser (those are instead powered by mains power).

In one embodiment, the circuit of FIG. 13 designed to operate from a +3VDC supply sourced by a single consumer-grade coin cell battery. Preferably, average power consumption of the circuit must be minimized to prolong battery life, thereby minimizing consumer maintenance issues and cost, and the battery should provide suitable supply voltage to operate for at least a few months.

The circuit of FIG. 13 comprises an analog comparator device. In one embodiment, an LM393 Low Power Dual Comparator may be used. Two independent comparator circuits are utilized to implement two separate functions in the battery-powered design: 1) sense ultralow current flow through oil-saturated wick of the dispenser; and 2) flash LED indicator to signal refill required. The circuit shown in FIG. 13 accomplishes both functions while simultaneously minimizing the average current consumption, both during the “sensing” mode as well as the “refill indication” mode.

The circuit operates from a single coin cell battery B1 that may be replaced by the consumer once the indicator circuit ceases to function. This may be evident when the LED fails to flash when the reservoir is removed. Removing the reservoir while the circuit is powered by a functional battery cell can result in the current sensing function failing, thereby triggering the multivibrator (pulse-generation) circuit. The multivibrator circuit is responsible for periodically flashing the LED, indicating the depletion of scented oil from the reservoir. The CR-2032 coin cell battery is readily available from numerous sources and is characterized by a suitable mAH (milli-Amp-Hour) capacity to power the circuit for an extended period of time.

One-half of the LM393 dual comparator U1 is used to sense the ultra-low current flowing through the wick contacted by CN1 and through the 10 megaohm resistor R5. Current flowing through R5 results in a voltage being applied to the non-inverting input of dual comparator at pin 3. U1A compares this voltage with the reference voltage at the inverting input at pin 2, which is set by the ratio of voltage divider resistors R1 and R6. If the input voltage exceeds the reference voltage, then the output of U1A is pulled high by resistor R2, which in turn disables transistor Q1. When transistor Q1 is off, no current flows through to the LED, and thus it is not illuminated. When the scented oil has sufficiently evaporated from the wick contacted by CN1, current ceases to flow through resistor R5, thereby reducing the voltage applied to pin 3 of U1A to zero volts. Since the input voltage now is less than the reference voltage at pin 2, the output of U1A is driven low which in turn enables transistor Q1. When transistor Q1 is on, +3V is applied to the anode of the LED.

The other half of the LM393 dual comparator U1 is configured as a multivibrator circuit, which generates a periodic low-going pulse. The width of this pulse as well as the frequency of this pulse is determined by the values of R8, R10, R11, and C1. Whenever the pulse occurs, the low signal causes current to flow through the LED, thereby illuminating the LED. By adjusting the values of the multivibrator circuit, the average current required during a refill indicator mode can be minimized. To use an LED with an integrated flash or blink capability, the multivibrator circuit may be eliminated and replaced by zero-ohm jumper R15.

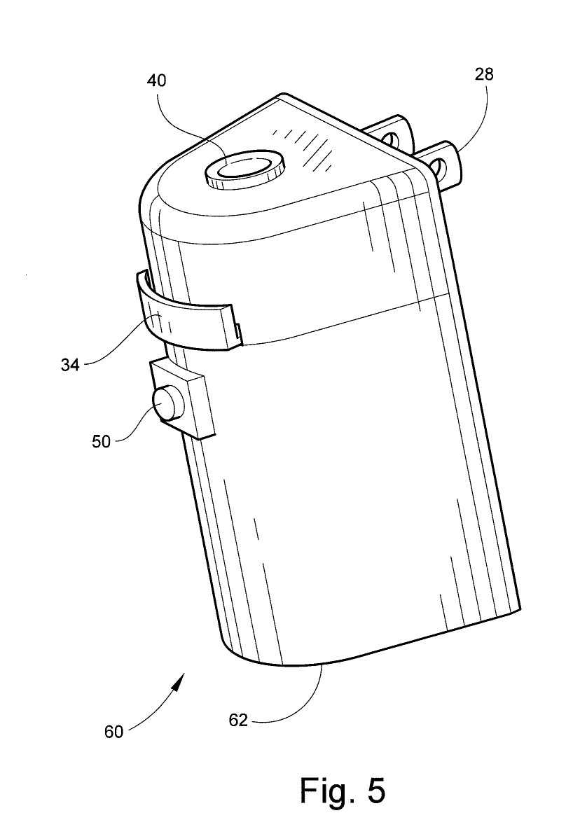

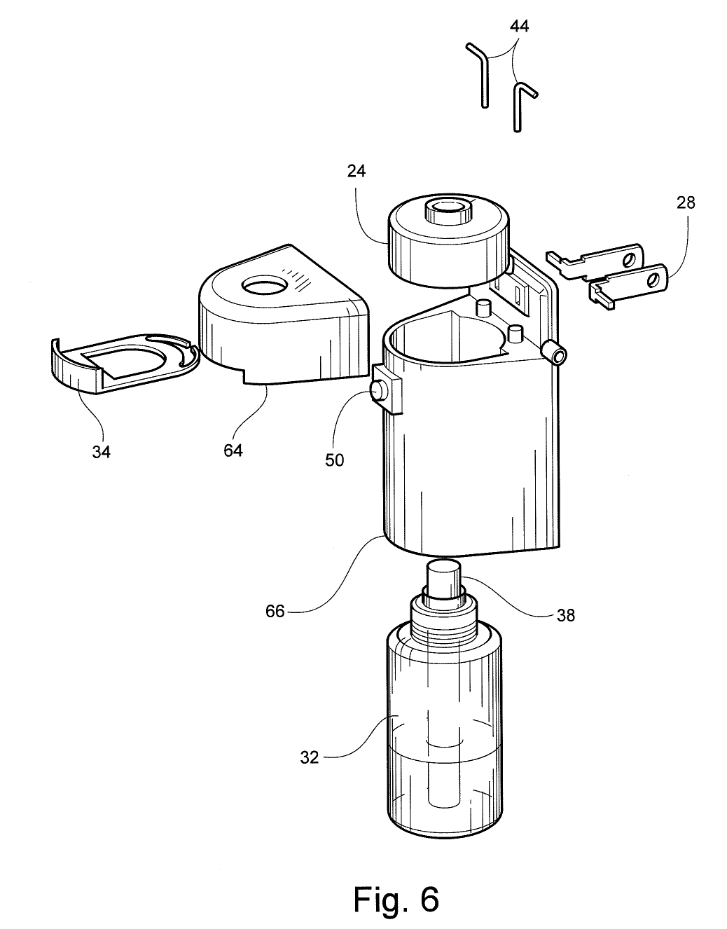

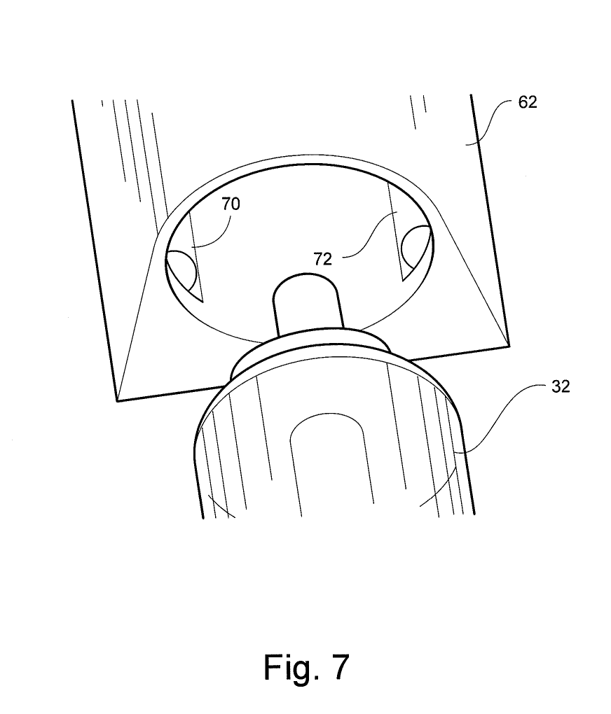

Another embodiment of a dispenser is shown in FIGS. 5-7. A dispenser 60 uses an electronic eye with an emitter 70 and a receiver 72, as shown in FIG. 7, to sense the level of vaporizable material in reservoir 32. Otherwise, dispenser 60 includes many of the same components described above with respect to FIGS. 1-4 and dispenser 20, as is clear from the drawings, including reservoir 32 with wick 38, plug 28, heating element 24, latch 34, and LED 50. A housing 62 is configured with a top portion 64 and a bottom portion 66, as shown in FIG. 6, and is slightly different than housing 22 shown in FIGS. 1-4.

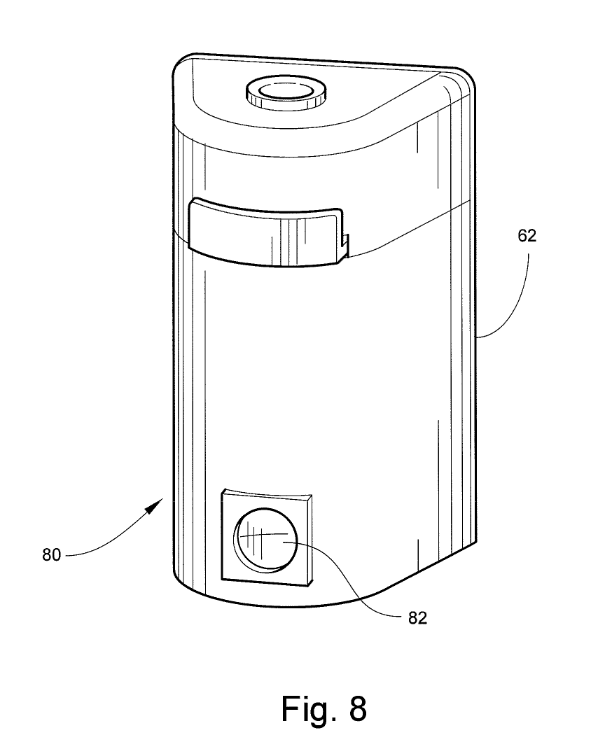

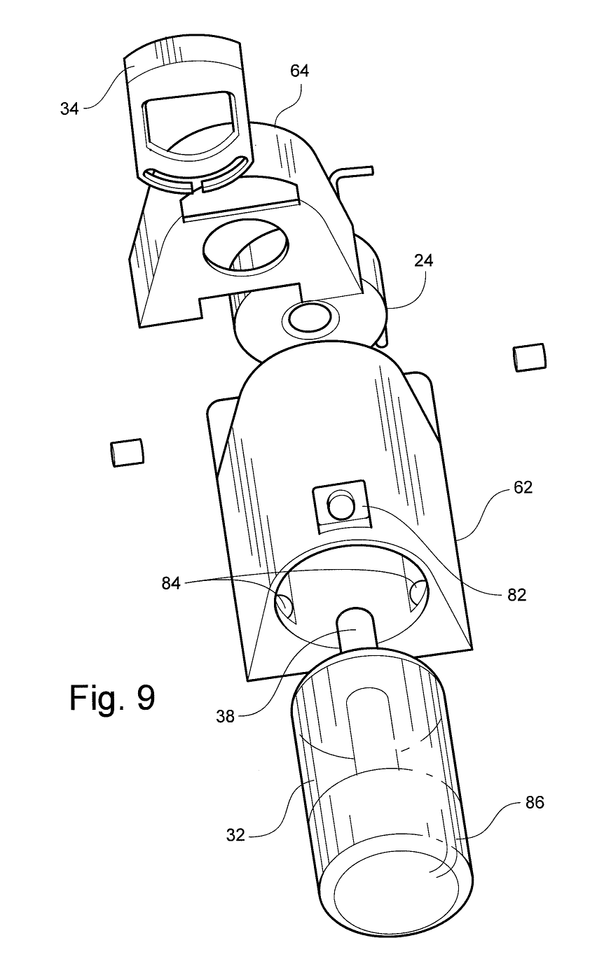

Another embodiment of a dispenser is shown in FIGS. 8 and 9. A dispenser 80 uses a window 82 in bottom portion 66 of housing 62 and lamps 84 positioned inside bottom portion 66. Lamps 84 remain on during use and shine through a colored liquid 86 in reservoir 32 and are visible through viewing window 82. When the liquid level is beneath the window level, the color seen through viewing window 82 changes. For example, if lamps 84 are clear and liquid 86 is blue, the color visible through window 82 changes from blue to clear to let the user know reservoir 32 is low or empty. Otherwise, dispenser 80 includes many of the same components described above with respect to FIGS. 1-4 and dispenser 20, as is clear from the drawings, including reservoir 32 with wick 38, plug 28, heating element 24, latch 34, and LED 50. It should be understood that the embodiments of dispensers shown in FIGS. 5-9 may be partially battery-powered using a circuit such as that shown in FIG. 13 or another suitable circuit, or powered entirely by battery.

Other embodiments of this invention include means other than an LED or similar lighted signal or an audible signal to indicate a low fluid level. For example, reservoirs designed specifically to emphasize the level of liquid in the reservoir, back lighting (electroluminescent or otherwise) to provide information about the amount of fluid in the reservoir, or using phosphorescent additives in the fluid are among some alternative embodiments. Many of the embodiments described below work passively, but are suitable for use with a circuit as a switch for an indicator.

In one embodiment, RFID tags are used. An RFID tag may be used within the housing or reservoir to sense vaporizable material, such as a liquid, in the reservoir or in the wick. For example, an RFID tag that is wet may reflect a certain signal when queried indicating that the reservoir has sufficient vaporizable material. When a change in wetness of the RFID tag occurs, such as when the RFID tag becomes dry or almost dry, the signal reflected may indicate that the reservoir is empty or almost empty and needs to be refilled or replaced. This may be particularly useful in a smart home or in conjunction with a business with high customer numbers that would deliver liquid refills for air fresheners or similar devices to a home or office as needed based on information received via the RFID tag.

In one embodiment, refraction and magnification is used. This embodiment may be used to increase visibility of the level of a vaporizable material by using the refractory effect that the vaporizable material has on light and the geometry of the reservoir to magnify this effect. For example, in one embodiment, the wick is visible when refracted by liquid in the reservoir. This technique may be used in conjunction with an electronic eye and incorporated as an electrical switch.

In another embodiment, a dispenser includes frosted glass to increase the visibility of the level of vaporizable material within the reservoir. When frosted glass is dipped in water, or some other type of liquid, for example, one can see through the frosted glass more easily. By using a glass reservoir and frosting the glass of the inner surface, or simply placing a piece of frosted glass vertically in the reservoir, the frosted glass assists the user in discerning whether or not a vaporizable material is present in the reservoir.

In another embodiment, a dispenser incorporates a fluid color filter that adds color to the vaporizable material in the reservoir. The colored fluid will allow the user to see more clearly whether or not the reservoir is empty simply by looking at the device such that the user is alerted that fluid is absent from the reservoir if the user does not see color. To further improve visibility, a backlight may be placed behind the reservoir.

In yet another embodiment, electroluminescent backlighting may be used to increase the visibility of the vaporizable material. In this embodiment, an electroluminescent strip is used to provide light behind the reservoir, thereby increasing the visibility of the vaporizable material, such as a liquid or gel. The color of the strip may be changed based on user preference, or perhaps designate a manufacturer's product line. In one embodiment, a white electroluminescent strip may be used with the fluid color filter described above.

In still yet another embodiment, blacklighting is used by introducing any variety of phosphorescent additives to the vaporizable material. When these additives come in contact with a black light, they glow. This increases the visibility of the vaporizable material in the reservoir such that the user knows that vaporizable material, such as a liquid or gel, is absent from the reservoir when there is an absence of a bright, glowing color.

In another embodiment, additives may be added to the wick such that the additives influence the natural properties of the liquid in the reservoir that is absorbed by the wick. For example, adding electrolytes may improve conductivity of the wick when wet with liquid such that conductivity, or some other electrical property, is more easily or better measured by a sensor within a dispenser.

In another embodiment, a water mirror may be used. A laser or other light source may be positioned to take advantage of the natural mirroring effects of surface water to light a diffuser to indicate when there is liquid in the reservoir.

In another aspect, the present invention provides methods of making a dispenser. A method of making a dispenser, in one embodiment, comprises providing a housing; coupling a reservoir to the housing, the reservoir containing a vaporizable material and a wick at least partially disposed in the reservoir; providing a circuit configured to measure a property in the wick or measure the vaporizable material in the reservoir; and coupling the circuit to an indicator operable to provide a signal if a measurement is above, below, or equal to a predetermined threshold.

In a further aspect, the present invention provides methods of indicating the level of a vaporizable material in a dispenser comprising providing a circuit; obtaining a measurement of a property in a wick of the dispenser or a measurement of the amount of vaporizable material in a reservoir of the dispenser with the circuit; coupling the circuit to an indicator; and providing a signal with the indicator based upon the value of the measurement. In some embodiments, the value of the measurement obtained by the circuit is greater than a predetermined threshold value. In other embodiments, the value is less than a predetermined threshold value. In a further embodiment, the value of the measurement obtained by the circuit is equal to a predetermined threshold value. In one embodiment, the signal is an audible and/or visual signal indicating that the level or amount of vaporizable material in the dispenser is low or depleted.

The foregoing description of embodiments of the invention has been presented only for the purposes of illustration and description and is not intended to be exhaustive or to limit the invention to the precise forms disclosed. Many modifications and variations are possible in light of the above teaching. The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope.

All patents, publications, and abstracts cited above are incorporated herein by reference in their entirety. It should be understood that the foregoing relates only to preferred embodiments of the present invention and that numerous modifications or alterations may be made therein without departing from the spirit and scope of the present invention as defined in the following claims.

1. A dispenser of vaporizable material, the dispenser comprising:
a housing;
a reservoir coupled to the housing, the reservoir containing a vaporizable material and a wick at least partially disposed within the reservoir;
a circuit configured to measure (a) a property in the wick or (b) the vaporizable material in the reservoir; and
an indicator triggered by the circuit if a measurement is above or below a predetermined threshold, the indicator providing a visual or audible signal indicating the level of the vaporizable material in the reservoir.

2. The dispenser of claim 1, further comprising a plug with contact blades extending from the housing.

3. The dispenser of claim 1, wherein the reservoir is removable from the housing.

4. The dispenser of claim 1, wherein the circuit further comprises an AC-DC power converter.

5. The dispenser of claim 1, wherein the indicator comprises a light source.

6. The dispenser of claim 5, wherein the light source comprises a light emitting diode that lights when a measurement is above or below the predetermined threshold.

7. The dispenser of claim 5, wherein the light source comprises a light emitting diode that changes color when a measurement is above or below the predetermined threshold.

8. The dispenser of claim 5, wherein the light source comprises a light emitting diode that blinks when a measurement is above or below the predetermined threshold.

9. The dispenser of claim 1, wherein the indicator comprises a sound card.

10. The dispenser of claim 1, further comprising a heating element positioned within the housing.

11. The dispenser of claim 1, further comprising a fan positioned within the housing.

12. The dispenser of claim 1, wherein the circuit further comprises three transistors configured in combination such that an emitter of a first transistor is coupled to a gate of a second transistor and an emitter of the second transistor is coupled to a gate of a third transistor.

13. The dispenser of claim 12, wherein the circuit further comprises an AC-DC power converter.

14. The dispenser of claim 1, wherein the circuit comprises an RFID tag for reflecting when a change in wetness in the RFID tag is detected.

15. The dispenser of claim 1, wherein the property measured in the wick comprises conductivity, capacitance, dielectric change, inductance, or temperature.

16. The dispenser of claim 1, further comprising a battery.

17. The dispenser of claim 1, wherein the circuit further comprises an analog comparator integrated circuit powered by a battery.

18. The dispenser of claim 1, further comprising a heating element or fan positioned within the housing, wherein the circuit and indicator are powered by a battery within the housing and the heating element or fan is configured to be powered by mains power from an electrical outlet.

19. The dispenser of claim 1, wherein the vaporizable material comprises a fragrance, deodorant, disinfectant, insect repellant, insecticide agent, or a combination thereof.

20. The dispenser of claim 1, wherein the vaporizable material is a liquid, gel, paste, or a solid.

21. A method of making a dispenser for vaporizable material comprising:
providing a housing;
coupling a reservoir to the housing, the reservoir comprising a vaporizable material and a wick at least partially disposed in the reservoir;
providing a circuit configured to measure (a) a property in the wick or (b) the vaporizable material in the reservoir; and
coupling the circuit to an indicator operable to provide a signal if a measurement is above or below a predetermined threshold.

22. A method of indicating the level of a vaporizable material in a dispenser comprising:
providing a circuit;
obtaining a measurement of (a) a property in a wick of the dispenser or (b) an amount of the vaporizable material in a reservoir of the dispenser with a circuit;
coupling the circuit to an indicator; and
providing a signal with the indicator based on the value of the measurement.
